(19) United States
(12) Patent Application Publication
(10) Pub. No.: US 2011/0301420 A1
(43) Pub. Date: Dec. 8, 2011

(54) Trauma Retractor

(76) Inventors: John P. Pryor, Moorestown, NJ (US); Carmela Calvo, Moorestown, NJ (US)

(21) Appl. No.: 13/055,403

(22) PCT Filed: Mar. 16, 2009
(86) PCT No.: PCT/US09/37279

§ 371 Date: Aug. 22, 2011

Related U.S. Application Data
(60) Provisional application No. 61/064,653, filed on Mar. 18, 2008.

(51) Int. Cl. A61B 1/32

(52) U.S. Cl. 600/207

(57) ABSTRACT

A trauma retractor optionally including deflation means and an enclosed pressurized vessel capable of being shaped as a torus. When the vessel is shaped as a torus and deflated, the vessel become rigid and one or more holes of the torus may enlarge or remain fixed in size.

### FIELD OF THE INVENTION

The invention relates to a surgical retractor. Specifically, the invention relates to a trauma retractor optionally including deflation means and an enclosed pressurized vessel capable of being shaped as a torus, whereby when the vessel is shaped as a torus and deflated, the vessel become rigid and one or more holes of the torus may enlarge or remain fixed in size.

### BACKGROUND OF THE INVENTION

Surgery is a complex task that requires optimal access and visualization of the tissue being dissected. Retraction of tissues to reveal an area of interest occupies a great deal of time and effort during an operation. Often additional personnel are required at the operating table whose only function is to retract the abdominal wall or a large tissue mass. In trauma surgery, efficient retraction is even more urgent. Life threatening bleeding in deep tissue is difficult to control without fast, easy, and durable retraction of tissue and subsequent access to the affected tissue.

Current retractors may include hand held retractors, (e.g., Richardson, Deaver, Malleable) free standing retractors, or retractors for attaching to the operating room table (e.g., Buckwalter, Balfour). Reference is made FIG. 1A and FIG. 1B, which are pictures of current trauma retractors. The trauma retractors system shown in FIG. 1A is developed by Omni-tract. The trauma retractors system shown in FIG. 1B is shown retracting an anatomical region.

Each of these trauma retractors are bulky, time consuming, require additional personnel to set up, and typically provide inadequate retraction of freely movable organs such as the bowel and lung. In addition, these trauma retractors are potentially damaging to the anatomical regions in which they retract. Each year, there are hundreds of injuries, such as tissue tear to internal organs resulting from the aggressive and prolonged retraction performed with these devices.

A need exists for a trauma retracting device that is safe, quick, and easy to use.

### SUMMARY OF THE INVENTION

In one embodiment, the invention provides a trauma retractor comprising: deflation means; and an enclosed pressurized vessel capable of being shaped as a torus, wherein when said vessel is shaped as a torus and deflated, said vessel becomes rigid and one or more holes of said torus enlarge or remain fixed in size.

In another embodiment, the invention provides a system comprising: a trauma retractor comprising a valve and an enclosed pressurized vessel capable of being shaped as a torus; and a suction device coupled to said valve for deflating said vessel, wherein when said vessel is shaped as a torus and deflated, said vessel becomes rigid and one or more holes of said torus enlarge or remain fixed in size.

In one embodiment, the invention provides a method for retracting an anatomical region within the body, the method comprising: shaping an enclosed pressurized vessel into a torus; and deflating said vessel, whereby when said vessel is shaped and deflated, said vessel becomes rigid and the size of one or more holes of said torus enlarge or remain fixed

### BRIEF DESCRIPTION OF THE DRAWINGS

Various embodiments of the present invention are illustrated in the following drawings, which are meant to be exemplary only and are not limiting on the scope of the present invention, and in which

FIG. 1A and FIG. 1B are pictures of current trauma retractors;

FIG. 2 is a schematic illustration of a trauma retractor system according to an embodiment of the invention;

FIG. 3 and FIG. 4 are pictures of examples of vacuum devices that may be used according to embodiments of the invention;

FIG. 5 is a schematic illustration of a flat vessel according to an embodiment of the invention;

FIG. 6 and FIG. 7 are schematic illustrations of vessels shaped as tori having one opening according to an embodiment of the invention;

FIG. 8 is a schematic illustration of a vessel shaped as a torus having two openings according to an embodiment of the invention;

FIG. 9 is a schematic illustration of a vessel shaped as a torus including a plurality of vessel bodies according to an embodiment of the invention;

FIG. 10 is a picture of an inflated trauma retractor prototype according to an embodiment of the invention;

FIG. 11 and FIG. 12 are pictures of an isometric view and a top view, respectively, of a deflated trauma retractor prototype according to an embodiment of the invention;

FIG. 13 is a picture of an example of a valve (8) that may be used according to embodiments of the invention; and

FIG. 14 is a picture of a motorized pressurization device (6) that may be used according to embodiments of the invention;

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DESCRIPTION OF THE PREFERRED EMBODIMENT

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

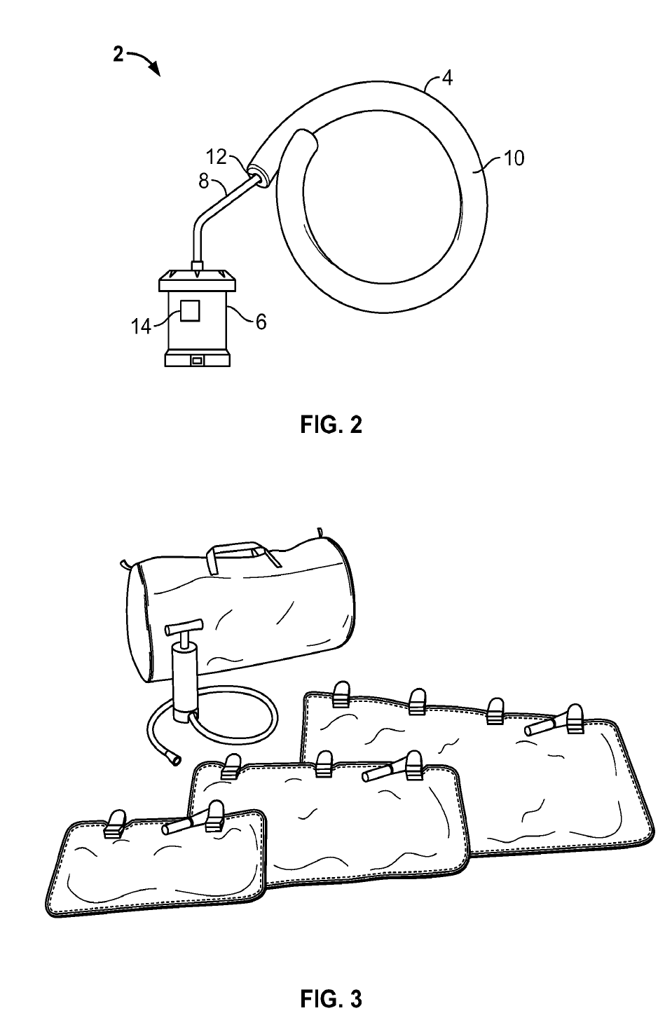

Reference is made to FIG. 2, which schematically illustrates a trauma retractor system 2 according to an embodiment of the invention. The system 2 may include a vessel 4, a pressurization device 6, and a valve 8 for providing a connection therebetween. Vessel 4 may include a housing 10 and malleable material (not shown) contained therein.

The pressurization device 6 may be adapted for changing the pressure of the vessel 4 (e.g., inside housing 10) via the connecting valve 8. The pressurization device 6 may include a motor or driving device 14 for activating changes in pressure, such as, suctioning (See e.g. FIG. 14). In one embodiment, pressurization device 6 may be used for deflating and inflating the vessel 4. In another embodiment, pressurization device 6 (e.g., a suction device) may only be used for deflating. In such embodiments, the vessel 4 may inflate by opening or releasing the valve 8 for equalizing the pressure internal to the vessel 4 and the pressure of the external environment thereof.

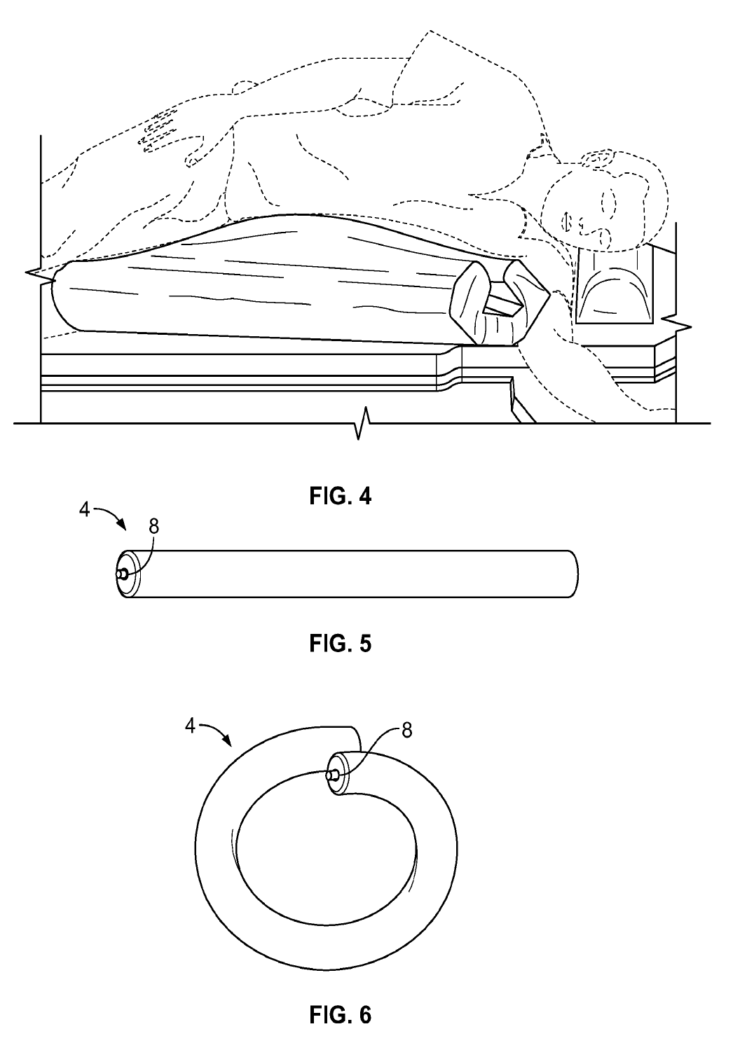

Pressurization device 6 may include a vacuum device coupled to the vessel 4 for inflating, deflating, suctioning or changing the pressure thereof. Reference is made to FIG. 3 and FIG. 4, which are pictures of examples of vacuum devices that may be used according to embodiments of the invention. The vacuum device in FIG. 3 is the Evac-U-Split®, manufactured by Hartwell Medical of Carlsbad, Calif. The vacuum device in FIG. 4 is typically used for splinting long bone fractures and positioning patients on the operating table. Other suction, deflation, inflation, vacuum, or pressurization devices may be used.

In one embodiment, vessel 4 may have a continuous range of pressures. In such embodiments, pressurization device 6 may be variably adjusted for applying the continuous range of pressures. For example, a user may select a pressure manually by adjusting a knob for tuning or a touch or dial pad for entering a desired pressure (not shown). In another embodiment, vessel 4 may have a few (e.g., two) discrete pressures (e.g., off/inflated and on/deflated). For example, vessel 4 may a user may punch a button or flip a switch (not shown) for activating vessel 4 deflation. In such embodiments, pressurization device 6 may automatically determine the optimal pressure required for making vessel 4 deflate based on the internal pressure thereof (e.g., using a pressure sensor). In another embodiment, for failsafe use, pressurization device 6 may include a non-motorized pump. Alternately vessel 4 may be pumped by a user blowing air into valve 8. In one embodiment, the pressurization device 6 is capable of both inflating and deflating the vessel 4.

The vessel 4 may be bendable or malleable for shaping when pressure inside the vessel is above a first threshold (e.g., when the vessel is inflated). The vessel 4 may be rigid when pressure inside the vessel is below a second threshold (e.g., when the vessel is deflated). Thus, the vessel 4 may be shaped in a high pressure or inflated state and then made rigid for use as a retractor in a low pressure or deflated state.

In one embodiment, for failsafe operation, once pressure is below a second predetermined threshold and the vessel 4 is made rigid, the vessel 4 may be sealed. Thus, if power were to fail, the vessel 4 may remain rigid.

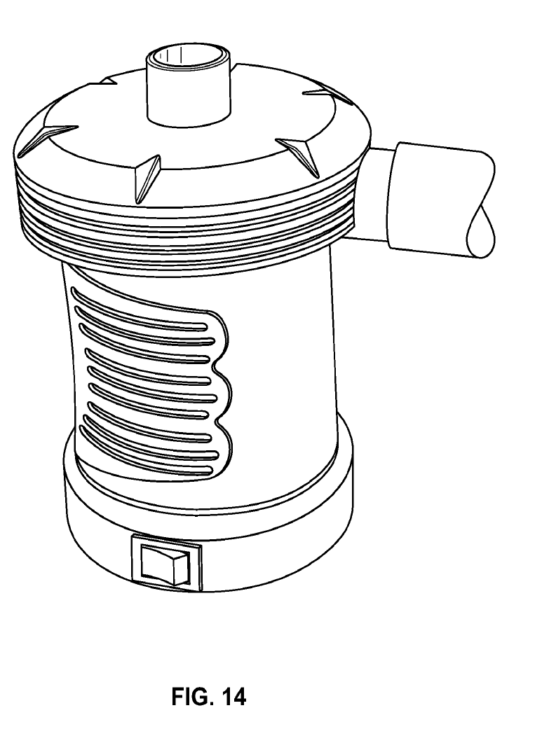

A shown in FIG. 13, the valve 8 may include a nozzle, hose, opening, or other means of connecting the vessel 4 and the pressurization device 6, for example, with an airtight seal. In one embodiment, the valve 8 may be permanently connected to the vessel 4 and/or the pressurization device 6. In another embodiment, the valve 8 may be detachably affixed to the structure, for example, via a mating element thereof. In such embodiments, the mating element may provide an airtight connection (e.g., by a sealing structure such as an adhesive film, clasps, plug, snaps, threading, or a combination thereof) between the valve 8 and the affixing structure.

The housing 10 may be a non-permeable or semi-permeable flexible membrane. For example, the housing 10 may be composed of materials such as plastics. The housing 10 may provide an airtight seal for enclosing the material. In one embodiment, as shown in FIG. 13, the housing 10 may have an opening 12, connecting the internal and external surfaces of the vessel 4, for example, for connecting the vessel 4 with the valve 8 or for equalizing pressure. In one embodiment, the opening 12 may be adapted for substantially containing the material internal to the vessel 4. For example, the opening 12 may be smaller than the material particles or the opening may include a screen or barrier (not shown) for preventing the release of the material from the vessel 4. The housing 10 may be composed of non-reactive material or may be coated with such. In one embodiment, housing 10 or an additional exterior changeable cover thereof may be disposable. In other embodiments, vessel 4 may be disposable. The material enclosed within the vessel 4 may include a formable or non-rigid material, such as, for example, sand, Styrofoam, gels, or other particles. Other materials may be used.

Reference is made to FIGS. 5-8, which schematically illustrate vessels shaped in various configurations according to embodiments of the invention. In one embodiment, the vessel may be shaped from an initially flat shape (e.g., shown in FIG. 5) into a torus (e.g., shown in FIGS. 6, 7, and 8), for example, by spiraling the vessel 4 body and connecting two ends thereof. The shaping or bending of vessel 4 is preferably changeable and reversible. In one embodiment, the two ends of vessel 4 may have a connecting surface, such as ridges, an adhesive layer, or other means for forming, sealing, or securing a connection therebetween.

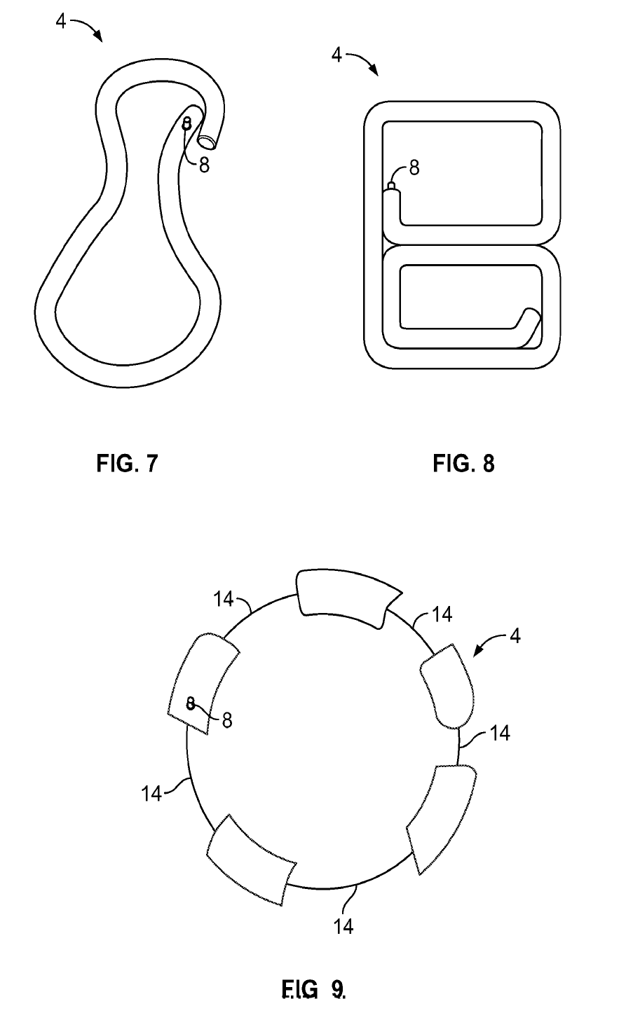

A torus may include any shape having at least one hole or opening therein. These shapes may include, for example, spheres, ellipsoids (e.g., as shown in FIGS. 4, 6, and 9), cubes, rectangular prisms (e.g., as shown in FIG. 8), n-sided prisms, polyhedron, each having at least one hole or opening. A torus may be a surface having a genus one. A genus may be a topological property of a surface defined as the largest number of nonintersecting simple closed curves that may be drawn on the surface without separating the surface. In general, the genus is the number of holes in a surface. A torus having one hole may be referred to, for example, as a “ring”, a “donut”, an “anchor ring” or simply “a” torus or “the” torus. A torus having a multiple number, n, holes may be referred to as an “n-torus”. For example, a torus having two holes may be referred to as a “2-torus” or a “double torus”; a torus having three holes may be referred to as a “3-torus” or a “triple torus”, and so on. FIGS. 6 and 7, schematically illustrate a vessel 4 shaped as a torus having one hole. FIG. 8 schematically illustrates a vessel 4 shaped as a torus having two holes.

In one embodiment, the vessel 4 may be a single enclosed structure, as shown in FIGS. 2 and 6-8. In another embodiment, shown in FIG. 9, the vessel 4 may include two or more separate vessel bodies having space therebetween and linked by a connecting material 16. In such embodiments, the connecting material 16 may be bendable for shaping the vessel 4, but substantially rigid for retracting an anatomical region. In both embodiments, the vessel 4 may be capable of being shaped as a torus.

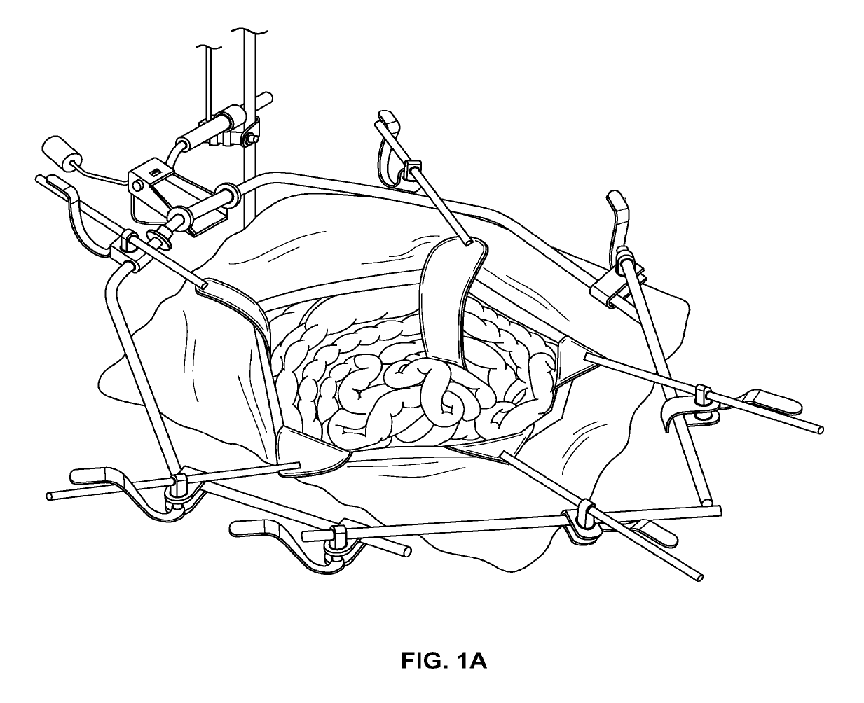

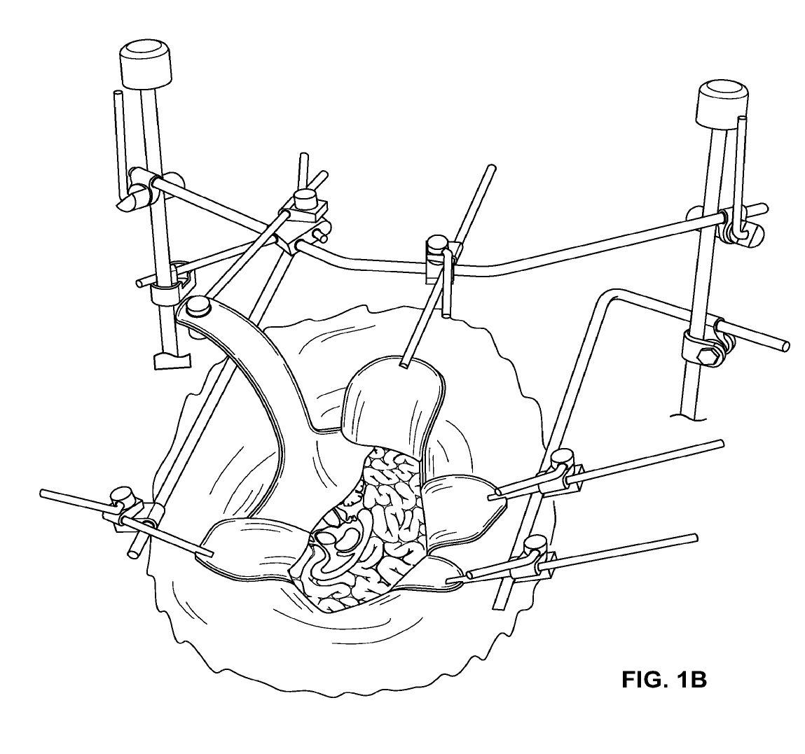

When used as a trauma retractor for retracting an anatomical region inside of a patient's body, the vessel 4 may preferably be shaped to fit the anatomical region to be retracted. Whereas conventional rigid trauma retractors provide minimal fitting (e.g., linking to several exterior locations at the periphery of the body opening as shown in FIG. 1A and FIG. 1B) vessel 4 may be custom molded (e.g., in a malleable inflated form) to mate with the contours of an anatomical body of substantially any shape in which the vessel 4 lies. In contrast to conventional retractors that pull on organs, when the retractor is deflated, the retractor effectively stents the organs and tissues away from the operative field of the body cavity, greatly decreasing the risk of injury. Since the trauma vacuum retractor is shapeable, the retractor may be used in substantially any body cavity. To fit relatively larger and relatively smaller anatomical regions, the vessel 4 may be shaped as a looser and tighter spiral, respectively (e.g., having a smaller and larger axis of rotation, respectively). The retractor typically has a substantially large surface area for securely positioning easily moveable or ‘floppy’ organs such as the bowel away from the operative field. The retractor may evenly distribute the forces of retraction. Vessel 4 may be customized to fit each anatomical region instantaneously, for example, during a surgery or procedure (e.g., in an intensive care unit, emergency department, or battlefield).

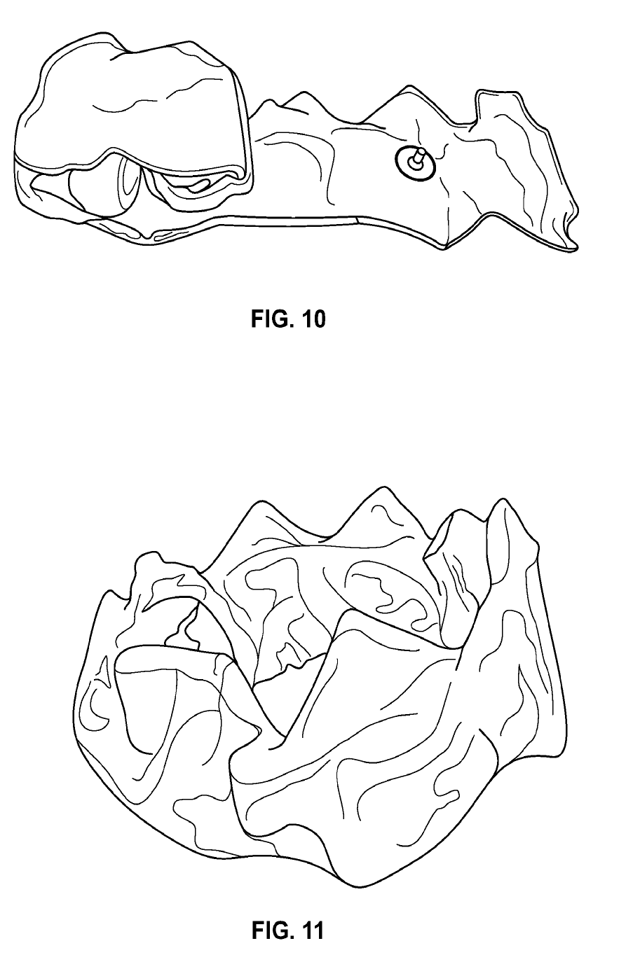

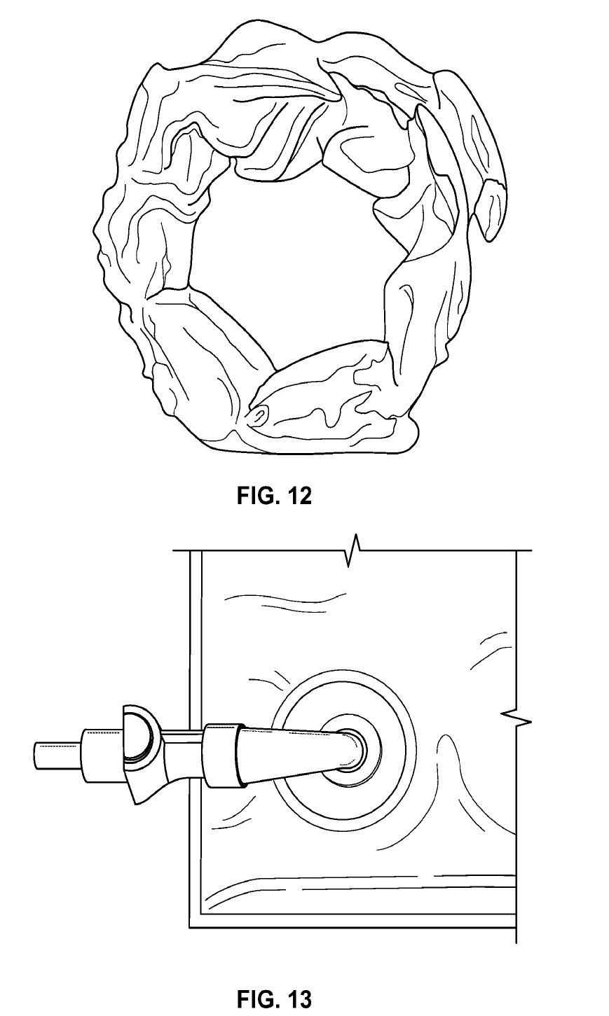

Reference is made to FIG. 10, which is a picture of an inflated trauma retractor prototype according to an embodiment of the invention. In this example, the trauma vacuum retractor is formed as a flat beanbag that may be shaped into a mold around an operative field of the body cavity. When suction is applied to the valve, a vacuum is created within the retractor that forces the particles in the retractor to coalesce and to become fixed and stiff. Thus, the retractor becomes deflated and rigid. Reference is made to FIG. 11 and FIG. 12, which are pictures of an isometric view and a top view, respectively, of a deflated trauma retractor prototype according to an embodiment of the invention.

The retractor devices described herein may reduce the time for retracting anatomical regions spent during operations, and may decrease the rate of inadvertent injury to internal organs. The retractor devices may be customizable to retract substantially any anatomical region, lightweight and disposable. Such retraction systems may forward surgical teams and combat support hospitals in the United States Military.

It is also contemplated that the trauma retractor described herein may be supplied in a kit containing the various parts. The kit may contain the pressurized vessel capable of being shaped as a torus, deflation means and instructions.

While the invention has been described with respect to a limited number of embodiments, it will be appreciated that many variations, modifications and other applications of the invention may be made. Embodiments of the present invention may include other apparatuses for performing the operations herein. Such apparatuses may integrate the elements discussed, or may comprise alternative components to carry out the same purpose. It will be appreciated by persons skilled in the art that the appended claims are intended to cover all such modifications and changes as fall within the true spirit of the invention.

What is claimed is:

1. A trauma retractor comprising:
deflation means; and
an enclosed pressurized vessel capable of being shaped as a torus, whereupon shaping said vessel is as a torus and its deflation, said vessel becomes rigid and one or more holes of said torus enlarge or remain fixed in size.

2. The trauma retractor of claim 1, further comprising inflation means, wherein when said vessel is shaped like a torus and inflated, said one or more holes of said torus reduce in size.

3. The trauma retractor of claim 1, wherein said vessel is malleable for shaping into said torus.

4. The trauma retractor of claim 1, wherein said vessel is shaped as said torus by spiraling a length of material of said vessel and overlapping two ends thereof.

5. The trauma retractor of claim 1, wherein said deflation means is detachably affixed to said vessel.

6. A system comprising:
a trauma retractor comprising a valve and an enclosed pressurized vessel capable of being shaped as a torus; and
a suction device coupled to said valve for deflating said vessel, wherein when said vessel is shaped as a torus and deflated, said vessel becomes rigid and one or more holes of said torus enlarge or remain fixed in size.

7. The system of claim 6, wherein said suction device is coupled to said valve for inflating said vessel and wherein when said vessel is shaped like a torus and inflated, said one or more holes of said torus reduce in size.

8. The system of claim 6, wherein said vessel is malleable for shaping into said torus.

9. The system of claim 6, wherein said vessel is shaped as said torus by spiraling a length of material of said vessel and overlapping two ends thereof.

10. The system of claim 6, wherein said valve is detachably affixed to said vessel.

11. A method for retracting an anatomical region within the body, the method comprising:
shaping an enclosed pressurized vessel into a torus; and
deflating said vessel, wherein when said vessel is shaped and deflated, said vessel becomes rigid and the size of one or more holes of said torus enlarge or remain fixed.

12. The method of claim 11, further comprising inflating said vessel, wherein when said vessel is shaped and inflated the size of said one or more holes of said torus reduce.

13. The method of claim 11, wherein said vessel is malleable for shaping into said torus.

14. The method of claim 11, wherein said shaping comprises spiraling a length of material of said vessel and overlapping two ends thereof.

15. The method of claim 14, wherein when said vessel is deflated said overlapping ends increase the rigidity of said vessel.
